(19) United States
(12) Patent Application Publication
(10) Pub. No.: US 2008/0003262 A1
(43) Pub. Date: Jan. 3, 2008

(54) COMPOSITIONS AND THERAPEUTIC METHODS OF USE

(75) Inventors: R. Saul Levinson, Chesterfield, MO (US); Robert C. Cuca, Glen Carbon, IL (US); Jonathan Bortz, St. Louis, MO (US)

Correspondence Address: KV Pharmaceutical Company, 4080B Wedgeway Court, Earth City, MO, 63045, US

(73) Assignee: DrugTech Corporation, Wilmington, DE (US)

(21) Appl. No.: 11/769,901

(22) Filed: Jun. 28, 2007

Related U.S. Application Data
(60) Provisional application No. 60/817,914, filed on Jun. 30, 2006.

(51) Int. Cl. A61K 31/7056; A61F 6/06; A61P 15/02

(52) U.S. Cl. 424/430; 514/24

(57) ABSTRACT

A method for treating non-infectious inflammatory vulvovaginitis comprises administering to a vulvovaginal surface a pharmaceutical composition that comprises clindamycin in an amount of about 125 mg to about 400 mg per unit dose of the composition; wherein the composition is bioadhesive to the vulvovaginal surface, and upon application of the composition to the vulvovaginal surface the clindamycin is released over a period of about 3 hours to about 14 days. A related method comprises administering to a vulvovaginal surface a pharmaceutical composition comprising clindamycin or a pharmaceutically acceptable salt or ester thereof, wherein the composition has at least one non-lipoidal internal phase and at least one lipoidal external phase that is bioadhesive to the vulvovaginal surface. A pharmaceutical composition comprises (a) clindamycin or a pharmaceutically acceptable salt or ester thereof in a clindamycin equivalent amount of about 2.5% to about 4% by weight; and (b) a phospholipid or non-ionic ester surfactant; wherein the composition is a vaginal cream having at least one non-lipoidal internal phase and at least one lipoidal external phase that is bioadhesive to a vaginal mucosal surface.

This application claims the benefit of U.S. provisional application Ser. No. 60/817,914, filed on Jun. 30, 2006, the entire disclosure of which is incorporated herein by reference.

### FIELD OF THE INVENTION

The present invention relates to vulvovaginally deliverable pharmaceutical compositions comprising clindamycin and therapeutic methods of use of such compositions.

### BACKGROUND OF THE INVENTION

Inflammatory disorders of the female urogenital system are underdiagnosed, often misdiagnosed and difficult to treat. This is especially true of inflammatory vulvovaginitis, an often painful and debilitating group of disorders which can occur at any stage of reproductive life and after menopause. Inflammatory vulvovaginitis can have infectious and non-infectious origins, though etiology is often difficult to establish as an opportunistic infection can hide a non-infectious origin of a particular disorder.

Non-infectious inflammatory vulvovaginitis includes disorders such as erosive vaginitis, idiopathic erosive vulvovaginitis, mucous membrane pemphigoid, lichen planus, lichen sclerosus, sterile vaginitis, vulvar vestibulitis, desquamative inflammatory vaginitis (DIV) and idiopathic inflammatory vaginitis. Symptoms vary among these disorders, but can include persistent purulent discharge, burning, soreness and dyspareunia; the vaginal epithelium typically is thin and bleeds easily. Physical distress and psychological distress coexist, and often women having non-infectious inflammatory vulvovaginitis are referred for psychosexual counseling.

Murphy (2004) Dermatologic Therapy 17(1):47-49 reports that there are no random double-blind controlled trials providing evidence for or against any particular treatment for DIV, though she mentions that topical steroids alone and in combination with clindamycin have been used.

Sobel (2003) Current Infectious Disease Reports 5(6):494-498 mentions that successful treatment of lichen sclerosus with clobetasol propionate is documented, and that topical tacrolimus is a valuable additive to therapy; that high potency fluorinated steroids are available as ointments, creams or gel preparations for treatment of erosive lichen planus; that DIV responds to intravaginal 2% clindamycin cream or 10% hydrocortisone; and that topical hydrocortisone, for example as a low-dose suppository, can be used to treat idiopathic erosive vulvovaginitis.

Sonnex (1999) Journal of Obstetrics and Gynaecology 19(1):41-43 reported variable results in treatment of vulvar vestibulitis with topical clindamycin cream.

Sobel (1994) Am. J. Obstet. Gynecol. 171(5):1215-1220 reported a retrospective study of patients with DIV who were treated with 2% topical clindamycin in suppository form. Clinical improvement was reportedly seen in more than 95% of the patients, though relapse was reported in 30%. It is mentioned that long term remission was obtained in a subset of patients when estrogen therapy was given.

U.S. Patent Application Publication No. 2004/0167223 of Popp relates to treatment of bacterial skin disorders with a mixture comprising about 0.9% to about 2.5% clindamycin and about 0.5% to about 20% benzoyl peroxide. DIV is among other disorders that, when associated with, related to or further occurring in skin affected by bacterial disorders, are said to be treatable with such a mixture.

International Patent Application Publication No. WO 2005/013906 of Maniar & Parandoosh proposes intravaginal delivery of actives using a pH responsive biocompatible film composition with a bioadhesive layer and an active agent. The publication also mentions that sustained release is preferred for some uses and that the composition can comprise a controlled release polymer. Clindamycin is listed among antibiotic agents said to be deliverable intravaginally using such a composition.

U.S. Pat. No. 4,551,148 to Riley et al. proposes a controlled release system for vaginal drug delivery, comprising unit cells having a nonlipoidal internal phase and a lipoidal continuous external phase. An active agent is present at least in the internal phase.

U.S. Pat. No. 5,266,329 to Riley proposes such a vaginal delivery system having an antifungal as the active agent.

Thompson & Levinson (2002) Drug Delivery Systems & Sciences 2(1):17-19 describe the VagiSite® bioadhesive topical drug delivery system as a high internal phase ratio water-in-oil emulsion system, providing a delivery platform for administration of active drug entities in the vaginal cavity. They disclose that the VagiSite® system is incorporated in Gynazole-1® antifingal vaginal cream.

U.S. Patent Application Publication No. 2003/0180366 of Kirschner et al. proposes a vaginal drug delivery system having globules comprising an internal water-soluble phase that is acid buffered and contains a drug, and an external water-insoluble phase or film.

U.S. Patent Application Publication No. 2004/0234606 of Levine et al. proposes a composition for vaginal administration comprising a treating agent (the tocolytic drug terbutaline is exemplified) and a bioadhesive cross-linked water-swellable but water-insoluble polycarboxylic acid such as polycarbophil, designed to give controlled and prolonged release of the drug through the vaginal mucosa. Administration of the composition is said to achieve local tissue concentrations without detrimental blood levels.

### SUMMARY OF THE INVENTION

There is now provided a method for treating non-infectious inflammatory vulvovaginitis, the method comprising administering to a vulvovaginal surface a pharmaceutical composition that comprises clindamycin or a pharmaceutically acceptable salt or ester thereof in an amount of about 125 mg to about 400 mg clindamycin equivalent per unit dose of the composition. The composition used in this method is bioadhesive to the vulvovaginal surface, and upon application of the composition to the vulvovaginal surface the clindamycin or salt or ester thereof is released over a period of about 3 hours to about 14 days.

There is further provided a method for treating non-infectious inflammatory vulvovaginitis, the method comprising administering to a vulvovaginal surface a pharmaceutical composition comprising clindamycin or a pharmaceutically acceptable salt or ester thereof. The composition used in this method has at least one non-lipoidal internal phase and at least one lipoidal external phase that is bioadhesive to the vulvovaginal surface.

There is still further provided a method for treating non-infectious inflammatory vulvovaginitis in a patient, the method comprising (a) administering to a vulvovaginal surface of the patient a pharmaceutical composition that comprises clindamycin or a pharmaceutically acceptable salt or ester thereof, wherein the composition is bioadhesive to the vulvovaginal surface; and (b) administering to the patient a pharmaceutical composition that comprises a steroid compound selected from the group consisting of corticosteroids and hormonal steroids.

There is still further provided a pharmaceutical composition in the form of a vaginal cream comprising (a) clindamycin or a pharmaceutically acceptable salt or ester thereof in a clindamycin equivalent amount of about 2.5% to about 4% by weight, and (b) a phospholipid or non-ionic ester surfactant; wherein the composition has at least one non-lipoidal internal phase and at least one lipoidal external phase that is bioadhesive to a vaginal mucosal surface.

There is still further provided a pharmaceutical composition in the form of a vaginal cream comprising (a) clindamycin or a pharmaceutically acceptable salt or ester thereof in a clindamycin equivalent amount of about 2.5% to about 8% by weight, and (b) a non-ionic ester surfactant; wherein the composition has at least one non-lipoidal internal phase and at least one lipoidal external phase that is bioadhesive to a vaginal mucosal surface, is bioadhesive to a vaginal mucosal surface, and is substantially free of phospholipid.

There is still further provided a pharmaceutical composition in the form of a vaginal cream comprising (a) clindamycin or a pharmaceutically acceptable salt or ester thereof in a clindamycin equivalent amount of about 2.5% to about 8% by weight, and (b) a steroid compound selected from the group consisting of corticosteroids and hormonal steroids; wherein the composition has at least one nonlipoidal internal phase and at least one lipoidal external phase that is bioadhesive to a vaginal mucosal surface.

There is still further provided a vaginal clindamycin delivery system comprising a unit dosage amount of a vaginal cream composition as described above and a disposable applicator therefor, wherein the applicator is prefilled with a unit dose amount of the composition.

Additional embodiments are described in the detailed description that follows.

### DETAILED DESCRIPTION

Provided herein are methods for treating non-infectious inflammatory vulvovaginitis. The phrase “non-infectious inflammatory vulvovaginitis” as used herein refers to any condition marked by vulvovaginal inflammation that is not primarily or evidently infectious in origin, or for which no causative infective agent has been identified at the priority date of the present application. By contrast, bacterial vaginosis, vulvovaginal candidiasis, and trichomonal vulvovaginitis are primarily infectious in origin and are associated with known infective agents. These conditions are therefore not considered herein to be forms of non-infectious inflammatory vulvovaginitis.

Non-infectious inflammatory vulvovaginitis includes but is not limited to erosive vaginitis, mucous membrane pemphigoid, lichen planus, lichen sclerosus, sterile vaginitis, vulvar vestibulitis, desquamative inflammatory vaginitis, idiopathic inflammatory vaginitis, autoimmune vaginitis and non-infectious inflammatory vulvovaginitis of unknown etiology. Pain, dyspareunia and postcoital burning are symptoms common to non-infectious inflammatory vulvovaginitis, and ulceration or blistering, i.e., a vaginal blistering disorder, can be but is not necessarily present. Desquamative inflammatory vaginitis (DIV), an illustrative non-infectious inflammatory vulvovaginitis, is characterized by diffuse exudative vaginitis, epithelial cell exfoliation, elevated vaginal pH, and increased levels of gram-positive cocci combined with complete or relative absence of normal long gram-positive bacilli. The DIV patient typically presents with discomfort, vaginal irritation and painful sexual intercourse.

In one embodiment, a method for treating non-infectious inflammatory vulvovaginitis, for example DIV, comprises administering to a vulvovaginal surface a pharmaceutical composition that comprises clindamycin or a pharmaceutically acceptable salt or ester thereof in an amount of about 125 mg to about 400 mg clindamycin equivalent per unit dose of the composition, wherein the composition is bioadhesive to the vulvovaginal surface, and upon application of the composition to the vulvovaginal surface the clindamycin or the pharmaceutically acceptable salt or ester thereof is released over a period of about 3 hours to about 14 days.

A “vulvovaginal surface” herein denotes any external or internal surface of the female genitalia, including mucosal surfaces in the vaginal cavity and nonmucosal surfaces of the vulva and immediately surrounding areas of skin. In some embodiments, the composition is more specifically adapted for application to a vaginal mucosal surface, and the external phase of the composition is bioadhesive to such a surface.

A composition used in methods of the invention can be in any suitable form that is adapted for vulvovaginal administration. For intravaginal administration, suitable forms include a vaginal cream, tablet, suppository, pessary or implant, but in particular embodiments, the composition is in the form of a vaginal cream.

The composition can be administered topically to external surfaces of the vulva and/or to surrounding areas of skin. In addition or alternatively, the composition can be administered intravaginally. In one embodiment, the composition is a vaginal cream, i.e., a semi-solid formulation adapted for administration to vaginal mucosal surfaces.

As used herein, except where the context demands otherwise, the term “clindamycin” refers to one or more of the compound clindamycin in its free base form and pharmaceutically acceptable salts and esters thereof, for example, clindamycin hydrochloride or clindamycin phosphate. Amounts of clindamycin are expressed herein as clindamycin (free base) equivalent amounts unless expressly indicated otherwise. A composition useful in the method of the present embodiment comprises about 125 to about 400 mg, for example about 150 to about 350 mg, about 125 to about 300 mg, about 125 to about 250 mg, or, illustratively, about 125 mg, about 150 mg, about 175 mg, about 200 mg, about 225 mg, about 250 mg, about 300 mg, about 350 mg or about 400 mg clindamycin equivalent per unit dose of the composition.

A unit dose is an amount of the composition suitable for a single administration to a vulvovaginal surface, for example a vaginal mucosal surface, as described herein. Most conveniently for the patient, the composition is provided in unit dose aliquots, typically individually packaged, but this is not a requirement of the present invention. Illustratively, a convenient unit dose aliquot of a vaginal cream is an amount of about 1 to about 10 g, although greater or lesser amounts, for example as little as about 0.1 or as much as about 25 g, can be used if desired. A particularly suitable unit dosage amount of a vaginal cream is about 2 to about 6 g, for example about 2 g, about 3 g, about 4 g or about 5 g. Where a unit dose is an amount of about 5 g, the total clindamycin equivalent concentration in the composition is suitably about 2.5% to about 8%, for example about 3% to about 7%, about 2.5% to about 6%, about 2.5% to about 5% or, illustratively, about 2.5%, about 3%, about 3.5%, about 4%, about 4.5% or about 5% by weight. Where the unit dose is greater or smaller than 5 g, suitable clindamycin equivalent concentration ranges will be correspondingly lower or higher respectively.

The amount of the composition to be administered will depend on the concentration of clindamycin in the composition, the frequency of administration (as determined for example by release rate) and other factors. As illustration, administration of 5 g of a composition containing 150 mg clindamycin equivalent, having a release period of 5 days, results nominally in delivery of 30 mg clindamycin equivalent on average per day. In practice, the amount of clindamycin delivered may be slightly lower than such a nominal amount, as it is often impracticable to ensure 100% delivery of a unit dose to the target surface.

Typically in a vaginal cream preparation a clindamycin equivalent amount of more than 2%, for example about 2.5% to about 8% by weight, or about 2.5% to about 6% by weight, for example about 2.5%, about 3%, about 3.5% or about 4% by weight, will be found useful.

A vaginal cream for use according to methods of the invention can be administered to contact a mucosal surface in the vaginal cavity by means, for example, of an applicator that is optionally pre-filled with a single unit dosage amount of the cream. With the patient optionally in a supine position, the tip of the applicator can be gently inserted high in the vagina, for example in the posterior vaginal formix, and the cream can be released through the tip by pushing on a plunger of the applicator.

Conveniently, a unit dosage amount of a vaginal cream for use according to the invention can be furnished in a prefilled container or applicator, for example an applicator similar to that used for Gynazole-1® vaginal cream of KV Pharmaceutical Co., St Louis, Mo. In some embodiments, the prefilled container or applicator is disposable.

Bioadhesion, for example to a vaginal mucosal surface, is an important property of compositions used according to the methods of the invention. It is believed, without being bound by theory, that bioadhesion allows for a sustained and controlled delivery of clindamycin or salts or esters thereof over time. Advantages over conventional vaginal delivery systems exhibiting less or no bioadhesion can include one or more of:
(a) minimization of leakage of the composition from the site of application;
(b) suitability for application at any time of day, not limited to bedtime;
(c) suitability for use during menses;
(d) reduction of active agent exposure, in particular systemic exposure, during a course of therapy;
(e) reduction of total active agent dose giving an acceptable clinical response;
(f) continuous active agent release during an extended period;
(g) more rapid relief of symptoms; and
(h) potential for single-dose therapy.

A “release period” or equivalent phrase herein refers to a period during which the clindamycin is made available for absorption and pharmacological effect at or close to the site of absorption, for example the vaginal cavity, in an amount sufficient to provide therapeutic benefit or prophylaxis with respect to a non-infectious inflammatory vulvovaginitis condition, for example DIV.

In the present embodiment, the composition is adapted for release of the clindamycin over a period of about 3 hours to about 14 days, for example, about 6 hours to about 10 days, about 12 hours to about 10 days, about 12 hours to about 7 days, about 1 to about 7 days, about 2 to about 7 days, or about 3 to about 7 days. In some embodiments, the composition is adapted for release of the clindamycin over a period consistent with a once daily to once weekly schedule, or a once to three times per week dosing schedule. In other embodiments, the composition is adapted for a twice monthly, or once monthly, dosing schedule or for intermittent use.

In a particular embodiment of the present method, a composition used according to the invention has at least one non-lipoidal internal phase and at least one lipoidal external phase that is bioadhesive to the vulvovaginal surface, as more fully described hereinbelow.

There is further provided a method for treating non-infectious inflammatory vaginitis, for example DIV, the method comprising administering to a vulvovaginal surface a pharmaceutical composition comprising clindamycin or a pharmaceutically acceptable salt or ester thereof, wherein the composition has at least one non-lipoidal internal phase and at least one lipoidal external phase that is bioadhesive to the vulvovaginal surface.

According to this method, the amount of clindamycin per unit dose is not limited but can usefully be about 75 to about 400 mg, for example about 100 to about 400 mg. In certain embodiments, the composition comprises, as in the previously described method, about 125 to about 400 mg, for example about 150 to about 350 mg, about 125 to about 300 mg, about 125 to about 250 mg, or, illustratively, about 125 mg, about 150 mg, about 175 mg, about 200 mg, about 225 mg, or about 250 mg clindamycin equivalent per unit dose of the composition.

Such a composition can illustratively take the form of a water-in-oil emulsion as generally described in any of above-referenced U.S. Pat. No. 4,551,148, U.S. Pat. No. 5,266,329 or U.S. Patent Application Publication No. 2003/0180366, or as further described in U.S. Patent Application Publication No. 2005/0095245 of Riley et al. Such a water-in-oil emulsion can be presented in a semi-solid form, for example as a vaginal cream.

In one embodiment, the composition is formulated as the VagiSite® bioadhesive topical drug delivery system described by Thompson & Levinson (2002), op. cit., or a delivery system substantially equivalent thereto, with inclusion of clindamycin as the active agent.

The clindamycin or salt or ester thereof can be present in either one or both of the internal and external phases. In one embodiment the clindamycin is present at least in part in the internal phase of the composition, and can be in dispersed form, for example in solution or suspension therein, or in non-dispersed form. Optionally, substantially all of the clindamycin can be present in the internal phase. Solubilization of the clindamycin can be achieved, for example, by use of a cosolvent and/or surfactant. The clindamycin can be present at least in part in particulate form, for example in micronized form, and can be dispersed as a particulate suspension in the internal and/or external phase. In various embodiments the clindamycin is present in solution, in aggregates, in liposomes, in microcapsules and/or in micelles within the internal and/or external phase.

A composition as described above having a non-lipoidal internal phase and a bioadhesive lipoidal external phase is, according to the present embodiment, adapted to release clindamycin over a suitable release period, for example, in one embodiment a period of about 3 hours to about 14 days, upon application to a vulvovaginal surface, for example a vaginal mucosal surface. Based on the disclosure herein, including disclosure of documents incorporated by reference herein, in particular above-referenced U.S. Pat. Nos. 4,551,148 and 5,266,329, and U.S. Patent Application Publication Nos. 2003/0180366 and 2005/0095245, one of skill in the art can without undue experimentation adjust release rate of clindamycin from such a composition to achieve a release period of about 3 hours to about 14 days as required according to the present embodiment. In various embodiments, the release period is one of about 6 hours to about 10 days, about 12 hours to about 10 days, about 12 hours to about 7 days, about 1 to about 7 days, about 2 to about 7 days, or about 3 to about 7 days. In particular embodiments, the release period is such that the composition is adapted for once daily to once weeldy administration, for example about 1 to about 3 times per week. In other embodiments, the composition is adapted for a twice monthly, or once monthly, dosing schedule or for intermittent use.

Release rate can be determined by in vivo testing or by any suitable in vitro method. An illustrative in vitro method utilizes an open chamber diffusion cell system such as a Franz cell system, typically fitted with an appropriate inert synthetic membrane such as polysulfone, cellulose acetate/nitrate mixed ester or polytetrafluoroethylene of suitable thickness, e.g., 70 μm. The receptor medium should be one in which the clindamycin is soluble, for example a water/ethanol medium. A test composition is placed uniformly on the membrane (illustratively, about 300 mg of a semi-solid composition such as a cream is a suitable amount for placement on a 25 mm diameter membrane) and is kept occluded to prevent solvent evaporation and compositional changes. This corresponds to an infinite dose condition. An aliquot of the receptor fluid is removed for analysis at appropriate intervals, and is replaced with an aliquot of fresh receptor fluid, so that the membrane remains in contact with the receptor fluid throughout the period of the release study. A release rate study such as that outlined above is typically replicated and can be conducted using a standard composition having known release properties for comparison.

The bioadhesive and sustained release properties of a vaginal cream used according to the invention can permit low frequency dosing of clindamycin to provide a clinically acceptable response, at least substantially equal to that provided by more frequent dosing of the active agent administered in the form of a conventional cream, for example, Cleocin®, a prescription vaginal cream containing 2% clindamycin as clindamycin phosphate. In particular, a single administration of a cream used according to the invention can provide a clinically acceptable response at least substantially equal to that provided by a conventional cream administered more than once, for example repeatedly about 3 to about 7 times in the course of one week. It is believed that the sustained release profile permitted by these compositions can provide therapeutically effective delivery at the locus of administration without substantially increasing systemic delivery.

The composition of a vaginal cream useful herein typically comprises a multiplicity of unit cells each having internal and external phases. The at least one internal phase can be discontinuous and is nonlipoidal and generally miscible with water. Illustratively, the internal phase comprises water, glycerin, propylene glycol, sorbitol or a combination of two or more thereof. The internal phase can itself be monophasic, biphasic or multiphasic, taking the form for example of a solution, suspension, emulsion or combination thereof. The internal phase can comprise one or more suspended solids, osmotic agents, extenders, diluents, buffers, chelating agents, preservatives or other materials.

Optionally, the internal phase is acid buffered to an internal pH of about 2.0 to about 6.0, for example about 2.5 to about 5.5 or about 3.5 to about 5.0. In one embodiment the internal phase is acid buffered to an internal pH that is substantially optimal to the vaginal environment, i.e., a pH that does not cause substantial irritation, itching or other discomfort and/or is detrimental to common pathogens of the vaginal cavity, including fungal pathogens such as Candida species and bacterial pathogens such as Enterococcus species. Typically such a pH is approximately 4.5.

The external phase is lipoidal and generally continuous. The term “lipoidal” herein can pertain to any of a group of organic compounds including neutral fats, fatty acids, waxes, phosphatides, petrolatum, fatty acid esters of monoprotic alcohols, mono-, di-, and triglyceride fatty acid esters, mineral oils, etc., typically having the following properties: insoluble in water; soluble in alcohol, ether, chloroform or other fat solvents; and exhibiting a greasy feel. Examples of suitable oils are mineral oils having viscosity of about 5.6 to about 68.7 centistokes, for example about 25 to about 65 centistokes, vegetable oils such as coconut, palm kernel, cocoa butter, cottonseed, peanut, olive, palm, sunflower, sesame, corn, safflower, rapeseed (canola) and soybean oils, and fractionated liquid triglycerides of naturally derived short-chain fatty acids.

The term “lipoidal” can also pertain to amphiphilic compounds, including for example natural and synthetic phospholipids. Suitable phospholipids can include, for example phosphatidylcholine esters such as dioleoylphosphatidylcholine, dimyristoyl-phosphatidylcholine, dipentadecanoylphosphatidylcholine, dipalmitoylphosphatidyl-choline (DPPC) and distearoylphosphatidylcholine (DSPC); phosphatidylethanolamine esters such as dioleoylphosphatidylethanolamine and dipalmitoylphosphatidylethanol-amine (DPPE); phosphatidylserine; phosphatidylglycerol; phosphatidylinositol; etc.

The term “lipoidal” can also pertain to certain amphiphilic compounds that are non-ionic esters including glyceryl and polyglyceryl esters of fatty acids, and ethoxylates of fatty acids or fatty acid esters. Others include sorbitan esters such as sorbitan monolaurate, sorbitan monopalmitate, sorbitan monostearate, sorbitan tristearate, sorbitan monooleate, sorbitan trioleate, sorbitan sesquioleate, and sorbitan monoisostearate.

Phospholipids and other amphiphilic compounds can enhance stability of the compositions used herein. In one embodiment, the external phase comprises a phospholipid component, for example, a lecithin component, more particularly a refined lecithin component. Without being bound by theory, it is believed that refined lecithins or other phospholipid materials can reside at the oil-water interface of a water-in-oil emulsion and impart improved stability to the emulsion, where an active agent such as clindamycin is present having surfactant properties that tend to disrupt emulsion stability. A preferred lecithin comprises not less than about 70%, for example, not less than about 80%, phosphatidylcholine. The phosphatidylcholine content of the lecithin can be as high as about 96% or even higher. Food grade lecithin is acceptable in some specific formulations but refined lecithins may be found preferable in other formulations. An example of a refined lecithin that is generally suitable is Phospholipon 90™, available from American Lecithin Co.

Amphiphilic non-ionic ester compounds can also act, optionally together with a phospholipid, as emulsifying agents in a composition of the invention. Any pharmaceutically acceptable emulsifying agent or combination thereof can be used, including without limitation medium and long chain monoglycerides and diglycerides, such as glyceryl monooleate, glyceryl monostearate, glyceryl monoisostearate and glyceryl monopalmitate, and polyglyceryl esters of fatty acids, such as polyglyceryl-3 oleate. Such agents can also function as emollients in the composition. Emulsifying agents soluble in the external phase are generally preferred. In one embodiment, a mono- and triglyceride mixture is used, alone or with addition of a metallic soap such as aluminum stearate.

Vaginal cream compositions comprising up to about 2% by weight clindamycin equivalent, as described, for example, in above-referenced U.S. Patent Application Publication No. 2005/0095245, can be useful in the present method. Typically, such compositions comprise at least about 1% by weight clindamycin equivalent. However, for certain types of non-infectious inflammatory vulvovaginitis such as DIV, there can be advantage in higher clindamycin concentrations, for example up to about 8%, typically about 2.5% to about 8%, more typically about 2.5% to about 4%, by weight.

Increased concentration of clindamycin equivalent above about 2% can present challenges in providing a physically stable vaginal cream formulation. It has been found that these challenges can be met through appropriate selection of amphiphilic compounds. Such selection can be made by one of ordinary skill in the art or by routine testing based on the disclosure herein. Thus, for example, a formulation having 2% clindamycin can be stabilized with a phospholipid and a non-ionic ester, but as clindamycin concentration increases, for example to 4%, improved stability can be achieved where the formulation comprises one or more non-ionic esters but a reduced amount or substantially no amount of phospholipid.

Water-in-oil emulsion compositions used herein are typically deformable at physiological temperatures (approximately 37° C.) but, unlike conventional creams, do not rapidly lose integrity upon application to a vaginal mucosal surface. In general, therefore, they do not result in offensive or otherwise unacceptable leakage from the vaginal cavity following administration. As physical breakdown of such compositions occurs over an extended period, nonaqueous components are either absorbed or released from the vaginal cavity at a generally unnoticeable rate, making no substantial increase over normal rates of vaginal secretion.

Release of the clindamycin from a composition as used according to the invention can occur by one or more mechanisms, none of which are limiting to the present invention. Such mechanisms can include diffusion, for example from the internal phase through the external phase into the vaginal mucosa; rupture of unit cells; dissolution of solid particulates; etc. Release dynamics can be linear or nonlinear.

Factors affecting release rate of the clindamycin can include the particular clindamycin equivalent used; the physical form of the clindamycin equivalent (e.g., whether in solution or in particulate form, and if particulate, average particle size); viscosity of the composition; selection and relative amounts of lipoidal compounds, including amphiphilic compounds, in the external phase; osmotic properties of the internal phase; and the relative volumes of the internal and external phases, among other factors. In a composition having the clindamycin in the internal phase, and having a relatively small internal phase ratio (expressed as percentage of total volume occupied by the internal phase), the external phase tends to form a relatively thick membrane through which the clindamycin must pass to be released; accordingly release rate can be significantly slowed in such a composition.

A suitable internal phase ratio can be established for any particular system by routine testing. Usually compositions used according to the invention are in the form of water-in-oil emulsions having medium to high internal phase ratio (expressed as percentage of total volume occupied by the internal phase), for example, greater than about 50%, greater than about 70%, or greater than about 75%, by volume. In one embodiment the internal phase ratio is at least about 70% by volume.

A semi-solid composition such as a vaginal cream used according to the methods of the invention can have a viscosity of about 5,000 to about 1,000,000 centipoise, for example about 100,000 to about 800,000 centipoise. In one embodiment the viscosity is about 350,000 to about 750,000 centipoise. In another embodiment the viscosity is about 100,000 to about 550,000 centipoise, for example about 250,000 to about 400,000 centipoise, or about 200,000 to about 350,000 centipoise. Bioadherence of a composition to a mucosal surface requires, among other properties, sufficient viscosity to retain integrity of the composition. Optional ingredients that can increase viscosity, among other properties, include microcrystalline wax, colloidal silicon dioxide, and various pharmaceutically acceptable polymers including polysaccharides, cellulosic polymers such as carboxymethylcellulose, methylcellulose, hydroxypropylmethylcellulose, etc., polyethylene glycol, acrylate polymers and the like.

In an illustrative embodiment, a vaginal cream comprises clindamycin or a pharmaceutically acceptable salt or ester thereof, for example clindamycin hydrochloride or clindamycin phosphate, water, sorbitol, propylene glycol, at least one long chain monoglyceride, for example glyceryl monooleate, glyceryl monostearate, glyceryl monoisostearate or glyceryl monopalmitate, a chelating agent, for example edetate disodium, at least one antimicrobial preservative, for example methylparaben and/or propylparaben, mineral oil, microcrystalline wax and colloidal silicon dioxide, for example hydrophobically modified colloidal silicon dioxide.

A particular example of a vaginal cream composition useful herein comprises clindamycin phosphate in a clindamycin equivalent amount of about 2.5%, about 3%, about 3.5% or about 4% by weight. The composition has (i) at least one nonlipoidal internal phase, (ii) at least one lipoidal external phase that is bioadhesive to a vaginal mucosal surface; and (iii) an emulsifying agent, for example comprising a non-ionic ester. The clindamycin phosphate is present at least in substantial part in the internal phase.

A composition such as that exemplified immediately above can be prepared by known batch or continuous processes for preparing pharmaceutical creams. As in preparing conventional emulsions, shear force is applied to the components by use of a mixer, homogenizer, mill, impingement surface, ultrasound, shaking or vibration. Mixing shear should be at a relatively low level to prevent destruction of the emulsion by excess energy.

Illustratively, the internal and external phases are first prepared separately. In a typical batch process, the internal phase is added to the external phase while mixing in a planetary-type or other suitable mixer until a stable emulsion is formed. Addition rates and mixing speeds can be adjusted to optimize formation and viscosity of the emulsion. In a typical continuous process, the external phase is introduced into a continuous mixer that comprises a plurality of impellers, until it reaches the level of the lowest impeller in the mixing chamber. The two phases are then simultaneously introduced through the bottom of the mixer in proper proportion as the impellers rotate to apply shear to the components. The finished emulsion emerges through the top of the mixer. Flow rate through the mixing chamber and mixing speed can be adjusted to optimize formation and viscosity of the emulsion.

A particular process of preparation is described in Example 1.

A prolonged release period is enabled by compositions used according to some embodiments of the invention. Such a prolonged release period brings a number of benefits to the patient, including without limitation those discussed immediately below.

First, frequency of application can be significantly reduced by comparison with a composition having faster release. In general, frequency of application of a prolonged-release composition for effective treatment can be once every 2 to 14 days, for example about 1 to about 3 times per week, illustratively about three times weekly, about twice weekly or about once weekly. In some embodiments, frequency of application is once per menstrual cycle, i.e., typically about once per month.

Second, the slow release from such a composition can result in maintenance of a therapeutically effective local concentration of clindamycin or salt or ester thereof without causing a major increase in systemic clindamycin (or equivalent) levels as measured, for example, by blood serum concentration. Risk of undesired or adverse side effects of increased serum clindamycin level is thus minimized.

Third, dosage amounts of the clindamycin can be reduced to levels close to the lowest effective dose for treatment of the non-infectious inflammatory vaginitis, for example DIV, taking advantage of the drug-sparing effect of slow release and minimizing adverse side effects.

Methods of the invention can involve repeated administration of a unit dosage amount of the composition until a clinically acceptable response is obtained; however, it is an advantage of at least some compositions of the invention over conventional vaginal creams that a clinically acceptable response is often obtainable with a single administration. A method wherein a single administration of a unit dosage amount provides a clinically acceptable response is often known as a “one dose to cure” therapy, but it will be recognized that the term “cure” in the present context does not necessarily mean total or permanent removal of the infection or total or permanent relief from all symptoms.

In one embodiment, a method of the invention provides, by a single administration, a “cure” rate at least substantially equal to that provided by about 3 to about 7 applications of a conventional vaginal cream composition containing 2% clindamycin.

In particular embodiments of the present method, a single dosage amount of about 2 g to about 6 g, for example about 2 g, about 3 g, about 4 g or about 5 g of a vaginal cream composition comprising clindamycin phosphate in a clindamycin equivalent amount of about 2.5%, about 3%, about 3.5% or about 4% by weight is administered to a vaginal mucosal surface. The composition has (i) at least one nonlipoidal internal phase, (ii) at least one lipoidal external phase that is bioadhesive to a vaginal mucosal surface; and (iii) an emulsifying agent, for example comprising a non-ionic ester. The clindamycin phosphate is present at least in substantial part in the internal phase.

In certain circumstances long-term remission from a non-infectious inflammatory vulvovaginitis condition such as DIV can be obtained by administration of a steroid compound in co-therapy with vulvovaginal administration of a clindamycin composition that is bioadhesive to a vulvovaginal surface as described herein. The steroid compound can have anti-inflammatory properties, as in the case of certain corticosteroids, that complement action of the clindamycin in treating the condition. Alternatively or in addition, the steroid compound can have hormonal, more particularly estrogenic, properties, that can enhance vulvovaginal health in various ways, for example by strengthening or thickening endothelial tissues and/or the mucosal lining of the vaginal tract. Such enhanced vulvovaginal health can, in some circumstances, reduce risk of recurrence of the condition.

Thus, in a still further embodiment, a method is provided for treating non-infectious inflammatory vulvovaginitis, for example DIV, the method comprising: (a) administering to a vulvovaginal surface a pharmaceutical composition that comprises clindamycin or a pharmaceutically acceptable salt or ester thereof, wherein the composition is bioadhesive to the vulvovaginal surface; and (b) administering a pharmaceutical composition that comprises a steroid compound.

The composition used in step (a) can be any bioadhesive clindamycin-containing composition, including but not limited to those described above having at least one non-lipoidal internal phase and at least one lipoidal external phase and/or comprising about 125 to about 400 mg clindamycin equivalent per unit dose.

The composition in step (b) can be administered by any suitable route, for example orally, transdermally, parenterally, subcutaneously, intravenously, intramuscularly, intraperitoneally, buccally, intravaginally, by inhalation, by depot injections, or by hormonal implants. In one embodiment the composition in step (b), like the composition in step (a), is administered intravaginally.

The composition administered in step (a) can be administered separately from, i.e., at a different time from, or together with, i.e., at substantially the same time as, the composition administered in step (b). In some embodiments, the composition in step (b) is adapted for vulvovaginal administration in a similar way to the composition in step (a), i.e., having properties promoting bioadhesion to a vulvovaginal surface, for example a vaginal mucosal surface. Thus, in one embodiment, each of the clindamycin and the steroid compound is independently provided in a composition that comprises at least one non-lipoidal internal phase and at least one lipoidal external phase that is bioadhesive to a vulvovaginal surface. For example, each of the clindamycin and the steroid compound can be independently provided in the form of a vaginal cream that is bioadhesive to a vaginal mucosal surface. In a particular embodiment, the clindamycin and the steroid compound are co-formulated in the same bioadhesive composition, for example a vaginal cream comprising both actives.

The steroid compound can be a corticosteroid or a hormonal steroid. Suitable corticosteroids include without limitation prednisone, prednisolone, cortisone, budesonide, hydrocortisone, dexamethasone, betamethasone, methylprednisolone and triamcinolone.

Suitable hormonal steroids include estrogenic compounds. An “estrogenic compound” herein is any steroidal compound or mixture thereof, whether of natural, biosynthetic or chemosynthetic origin, having estrogenic activity in a human female. Illustrative steroidal estrogenic compounds include without limitation conjugated estrogenic hormones (e.g., Premarin®), equilenin, equilin, estradiol, estriol, estrone, ethinyl estradiol, mestranol, moxestrol, quinestradiol, quinestrol, derivatives such as salts and esters thereof, enantiomers and racemates thereof, mixtures thereof and the like.

Where the clindamycin and the steroid compound are co-formulated in a single bioadhesive composition adapted for vulvovaginal administration and having non-lipoidal internal and lipoidal external phases, the clindamycin, the steroid compound or both can be present in either one or both of the internal and external phases. In one embodiment both agents are present at least in part in the internal phase of the composition, and can be in dispersed form, for example in solution or suspension therein, or in non-dispersed form. Optionally, substantially all of the clindamycin and/or substantially all of the steroid compound can be present in the internal phase. Solubilization of one or both agents can be achieved, for example, by use of a cosolvent and/or surfactant. Some agents, for example clindamycin phosphate, are fairly water soluble or readily solubilized, and such agents are typically present at least in part in solution in the internal phase. Commonly, however, one or both agents can be present at least in part in particulate form, for example in micronized form or in nanoparticulate form, and can be dispersed as a particulate suspension in the internal and/or external phase. In various embodiments the clindamycin, the steroid compound or both are present in aggregates or liposomes within the internal and/or external phase.

In compositions having one or both active agents in solid particulate form, any suitable particle size can be used. Typically, however, good physical stability can be difficult to achieve where a substantial portion of the particles are greater than about 250 μm in diameter. Thus a D₉₀ particle size (wherein 90% by weight of the particles are smaller than the specified size) not greater than about 250 μm is generally desirable for both the clindamycin and steroid compound. Preferably at least 99% by weight of the particles are not greater than about 250 μm in diameter.

Particle sizes smaller than about 5 μm can be useful but the expense of particle size reduction may not be justified by any improvement in stability or efficacy at such particle sizes. Nonetheless, particle sizes as small as 0.4 μm (400 nm), or even as small as 50 nm, can be used if desired.

Certain compositions described hereinabove as being useful in methods of the invention are themselves embodiments of the invention.

In one such embodiment, there is provided a pharmaceutical composition in the form of a vaginal cream comprising (a) clindamycin or a pharmaceutically acceptable salt or ester thereof in a clindamycin equivalent amount of about 2.5% to about 4% by weight; and (b) a phospholipid or non-ionic ester surfactant; wherein the composition is a vaginal cream having at least one non-lipoidal internal phase and at least one lipoidal external phase that is bioadhesive to a vaginal mucosal surface.

For example, the lipoidal external phase can comprise at least one non-ionic ester surfactant. Illustrative non-ionic esters include but are not limited to glyceryl and polyglyceryl esters of fatty acids, and ethoxylates of fatty acids or fatty acid esters. Others include sorbitan esters such as sorbitan monolaurate, sorbitan monopalmitate, sorbitan monostearate, sorbitan tristearate, sorbitan monooleate, sorbitan trioleate, sorbitan sesquioleate and sorbitan monoisostearate.

Alternatively or in addition, the lipoidal external phase can comprise at least one phospholipid as more fully described above. In an illustrative composition of the present embodiment, one or more phospholipids are present in an amount of 0% to about 2%, for example 0% to about 1% or 0% to about 0.5%, by weight of the total composition.

A pharmaceutical composition of another embodiment of the invention comprises (a) clindamycin or a pharmaceutically acceptable salt or ester thereof in a clindamycin equivalent amount of about 2.5% to about 8% by weight; and (b) a non-ionic ester surfactant; wherein the composition is a vaginal cream having at least one non-lipoidal internal phase and at least one lipoidal external phase that is bioadhesive to a vaginal mucosal surface. The composition of this embodiment is substantially free of phospholipid.

A pharmaceutical composition of yet another embodiment of the invention comprises (a) clindamycin or a pharmaceutically acceptable salt or ester thereof in a clindamycin equivalent amount of about 2.5% to about 8% by weight; and (b) a steroid compound selected from the group consisting of corticosteroids and hormonal steroids. The composition is a vaginal cream having at least one nonlipoidal internal phase and at least one lipoidal external phase that is bioadhesive to a vaginal mucosal surface.

In yet further embodiments, there is provided a vaginal clindamycin delivery system comprising a unit dosage amount of a vaginal cream composition of any of the embodiments hereinabove described, and a disposable applicator therefor, wherein the applicator is prefilled with a unit dose amount of the composition.

In one such embodiment, the composition comprises clindamycin or a pharmaceutically acceptable salt or ester thereof in a clindamycin equivalent amount of about 2.5% to about 4% by weight and has at least one nonlipoidal internal phase and at least one lipoidal external phase that is bioadhesive to a vaginal mucosal surface.

### EXAMPLES

The following examples are merely illustrative, and do not limit this disclosure in any way.

“USP” refers to U.S. Pharmacopoeia; “NF” refers to National Formulary.

Each of the compositions detailed below can be prepared by any method known in the art for preparing semi-solid emulsions, including batch and continuous processes as described hereinabove.

### Example 1

Purified water, sorbitol solution and edetate disodium were loaded into a stainless steel mixing tank equipped with a cover and variable speed mixer and mixed at room temperature until all solids were dissolved, to prepare an aqueous phase solution. If buffers such as citrate salts were used, they were added to the mixture and dissolved. Clindamycin phosphate was added to the aqueous phase solution and mixed until dissolved.

An oil phase solution was prepared by mixing, at about 70° C. to about 75° C., mineral oil, polyglyceryl-3-oleate, glyceryl monoisostearate and microcrystalline wax in a stainless steel jacketed kettle equipped with a sweep blade and variable speed mixer. When all solids were dissolved, a small portion of the oil phase was placed in a smaller stainless steel container and Phospholipon 90G was added. The mixture was stirred at about 80° C. to about 85° C. until the Phospholipon 90G was completely dissolved. The resulting Phospholipon 90G solution was added back to the oil phase solution; methylparaben and propylparaben were then added to the oil phase solution and dissolved at about 70° C. to about 75° C. Hydrophobic silicon dioxide was added to the oil phase solution and mixed to create an initial dispersion. While mixing, the oil phase solution was transferred through a colloid mill into a stainless steel jacketed kettle equipped with a counter rotation blade and a sweep blade.

The oil and aqueous phases were combined by adding the aqueous phase in a controlled manner to the oil phase solution with mixing until aqueous phase addition was complete. Mixing was continued for a period of time to establish a preliminary emulsion. The preliminary emulsion was transferred by transfer pump through a secondary mixing chamber at pre-established flow rates and mixing speeds in order to achieve final viscosity. The resulting vaginal cream was then transferred into bulk containers for packaging into individual vaginal applicators.

Table 1 presents ingredients and weight percentages of a formulation prepared according to the process described above. The amount of active ingredient and water to be added was calculated per batch based upon the assay and water content of the raw materials.

TABLE 1
Clindamycin 2% vaginal cream formulation

| Ingredient | Wt % |
|---|---|
| water, purified, USP | 45.30 |
| sorbitol solution | 36.80 |
| edetate disodium, USP | 0.05 |
| clindamycin phosphate, USP | 2.80 |
| mineral oil, USP | 7.00 |
| polyglyceryl-3-oleate | 2.70 |
| glyceryl monoisostearate | 2.70 |
| lecithin (Phospholipon ™ 90G) | 1.00 |
| silicon dioxide, hydrophobic | 1.00 |
| microcrystalline wax, NF | 0.40 |
| methylparaben, NF | 0.20 |
| propylparaben, NF | 0.05 |

Viscosity of the formulation based on in-process measurement was 860,000 cPs.

### Example 2

A vaginal cream formulation was prepared substantially according to the procedure described in Example 1. Table 2 presents ingredients and weight percentages of the formulation.

TABLE 2
Clindamycin 2% vaginal cream formulation

| Ingredient | Wt % |
|---|---|
| water, purified, USP | 41.98 |
| sorbitol solution | 39.60 |
| edetate disodium, USP | 0.05 |
| clindamycin phosphate, USP | 2.69 |
| mineral oil, USP | 10.00 |
| PEG-30 dipolyhydroxystearate | 5.00 |
| microcrystalline wax, NF | 0.43 |
| methylparaben, NF | 0.18 |
| propylparaben, NF | 0.05 |

Initial viscosity of the formulation was 224,000 cPs.

### Example 3

A vaginal cream formulation was prepared substantially according to the procedure described in Example 1. Table 3 presents ingredients and weight percentages of the formulation.

TABLE 3
Clindamycin 2% vaginal cream formulation

| Ingredient | Wt % |
|---|---|
| water, purified, USP | 45.23 |
| sorbitol solution | 30.00 |
| edetate disodium, USP | 0.25 |
| clindamycin phosphate, USP | 2.69 |
| mineral oil, USP | 8.00 |
| sorbitan monoisostearate | 8.00 |
| sorbitan monostearate | 4.00 |
| silicon dioxide, hydrophobic | 1.00 |
| microcrystalline wax, NF | 0.60 |
| methylparaben, NF | 0.18 |
| propylparaben, NF | 0.05 |

Initial viscosity of the formulation was 400,000 cPs.

### Example 4

A vaginal cream formulation (Table 4) containing citrate buffered clindamycin is prepared substantially according to the procedure described in Example 1.

TABLE 4
Citrate buffered clindamycin 2% vaginal cream formulation

| Ingredient | Wt % |
|---|---|
| water, purified, USP | 45.30 |
| sorbitol solution | 36.10 |
| edetate disodium, USP | 0.05 |
| citric acid USP, anhydrous | 0.49 |
| potassium hydroxide | 0.24 |
| clindamycin phosphate, USP | 2.80 |
| mineral oil, USP | 7.00 |
| polyglyceryl-3-oleate | 2.70 |
| glyceryl monoisostearate | 2.70 |
| lecithin (Phospholipon ™ 90G) | 1.00 |
| silicon dioxide, hydrophobic | 1.00 |
| microcrystalline wax, NF | 0.40 |
| methylparaben, NF | 0.20 |
| propylparaben, NF | 0.05 |

### Example 5

This example demonstrates a vaginal cream formulation having higher clindamycin concentration (4%) and lacking phospholipid (Table 5).

TABLE 5
Clindamycin 4% vaginal cream formulation

| Ingredient | Wt % |
|---|---|
| water, purified, USP | 38.89 |
| sorbitol solution | 37.30 |
| edetate disodium, USP | 0.50 |
| clindamycin phosphate, USP | 5.61 |
| mineral oil, USP | 10.00 |
| PEG-30 dipolyhydroxystearate | 4.00 |
| hydrogenated vegetable oil, NF | 1.50 |
| glyceryl monoisostearate | 2.00 |
| microcrystalline wax, NF | 0.40 |
| methylparaben, NF | 0.20 |
| propylparaben, NF | 0.05 |

The weight percentages for water and clindamycin phosphate are theoretical values providing 40.0 mg/g clindamycin equivalent, based on a minimum USP specification for potency and correcting for maximum allowed percent water. The amounts of these ingredients used can be adjusted based on actual potency and water content.

### Example 6

This example (Table 6) demonstrates four vaginal cream formulations 6.1-6.4, each containing 4% clindamycin, that were less desirable than that of Example 5 due to low viscosity or phase separation.

TABLE 6
Clindamycin 4% vaginal cream formulations

| | 6.1 | 6.2 | 6.3 | 6.4 |
|---|---|---|---|---|
| Ingredients | Wt % | Wt % | Wt % | Wt % |
| water | 43.81 | 42.81 | 40.11 | 42.16 |
| sorbitol solution 70% | 36.19 | 36.19 | 33.49 | 35.54 |
| edetate disodium, USP | 0.05 | 0.05 | 0.05 | 0.05 |
| clindamycin phosphate | 4.906 | 4.906 | 4.906 | 4.906 |
| Gloria mineral oil, USP | 7.00 | 7.00 | 7.00 | 7.00 |
| polyglycerol-3 oleate | 2.70 | 2.70 | 5.40 | 0 |
| Phospholipon ™ 90G | 1.00 | 2.00 | 2.00 | 2.00 |
| hydrophobic silicon dioxide (R972) | 1.00 | 1.00 | 1.00 | 1.00 |
| microcrystalline wax, w-835 | 0.40 | 0.40 | 0.40 | 0.40 |
| methylparaben, NF | 0.20 | 0.20 | 0.20 | 0.20 |
| propylparaben, NF | 0.05 | 0.05 | 0.05 | 0.05 |
| glyceryl isostearate | 2.70 | 2.70 | 5.40 | 2.70 |
| PEG 30 dipolyhydroxystearate | 0 | 0 | 0 | 4.00 |

### Example 7

Vaginal cream formulations (Table 7) containing clindamycin at 2-4% are prepared substantially according to the procedure described in Example 1.

TABLE 7
Clindamycin vaginal cream formulations

| Ingredient | Wt % | | | | |
|---|---|---|---|---|---|
| Clindamycin (target %): | 2 | 2.5 | 3 | 3.5 | 4 |
| water, purified, USP | 52.6 | 52.9 | 52.2 | 51.5 | 50.8 |
| sorbitol solution | 36.0 | 36.0 | 36.0 | 36.0 | 36.0 |
| clindamycin phosphate, USP | 2.8 | 3.5 | 4.2 | 4.9 | 5.6 |
| PEG-30 dipolyhydroxystearate | 4.0 | 4.0 | 4.0 | 4.0 | 4.0 |
| glyceryl monoisostearate | 2.0 | 2.0 | 2.0 | 2.0 | 2.0 |
| lecithin | 1.0 | 0.0 | 0.0 | 0.0 | 0.0 |
| silicon dioxide, hydrophobic | 1.0 | 1.0 | 1.0 | 1.0 | 1.0 |
| microcrystalline wax, NF | 0.6 | 0.6 | 0.6 | 0.6 | 0.6 |

All patents and publications cited herein are incorporated by reference into this application in their entirety.

The words “comprise”, “comprises”, and “comprising” are to be interpreted inclusively rather than exclusively.

What is claimed is:

1. A method for treating non-infectious inflammatory vulvovaginitis, the method comprising administering to a vulvovaginal surface a pharmaceutical composition that comprises clindamycin or a pharmaceutically acceptable salt or ester thereof in an amount of about 125 mg to about 400 mg clindamycin equivalent per unit dose of the composition; wherein the composition is bioadhesive to the vulvovaginal surface, and upon application of the composition to the vulvovaginal surface the clindamycin or salt or ester thereof is released over a period of about 3 hours to about 14 days.

2. The method of claim 1, wherein the composition comprises at least one non-lipoidal internal phase and at least one lipoidal external phase.

3. The method of claim 1, wherein upon application of the composition to the vulvovaginal surface the clindamycin or pharmaceutically acceptable salt or ester thereof is released over a period of about 2 to about 14 days.

4. The method of claim 3, wherein the clindamycin or pharmaceutically acceptable salt or ester thereof is released over a period consistent with a once daily to once monthly dosing schedule.

5. The method of claim 3, wherein the clindamycin or the pharmaceutically acceptable salt or ester thereof is released over a period consistent with a once to three times per week dosing schedule.

6. The method of claim 1, wherein the composition is administered at a frequency of about 1 to about 3 times per week.

7. The method of claim 1, wherein the composition is administered in a single dosage amount effective to provide an acceptable clinical response.

8. The method of claim 1, wherein the clindamycin is in the form of clindamycin phosphate.

9. The method of claim 1, wherein the composition comprises clindamycin or a pharmaceutically acceptable salt or ester thereof in a clindamycin equivalent amount of about 125 mg to about 200 mg per unit dose of the composition.

10. The method of claim 1, wherein the vulvovaginal surface to which the composition is administered is a vaginal mucosal surface.

11. The method of claim 10, wherein the composition is in a form of a vaginal cream having at least one non-lipoidal internal phase and at least one lipoidal external phase that is bioadhesive to the vaginal mucosal surface.

12. The method of claim 11, wherein the vaginal cream is administered with the aid of a disposable applicator that is prefilled with a unit dose amount of the vaginal cream.

13. The method of claim 11, wherein the vaginal cream comprises clindamycin or a pharmaceutically acceptable salt or ester thereof in a clindamycin equivalent amount of about 2.5% to about 8% by weight.

14. The method of claim 11, wherein the vaginal cream comprises clindamycin or a pharmaceutically acceptable salt or ester thereof in a clindamycin equivalent amount of about 2.5% to about 4% by weight.

15. The method of claim 14, comprising administering to a vaginal mucosal surface a unit dosage amount of about 2 to about 6 g of the vaginal cream.

16. The method of claim 11, wherein a unit dose of the vaginal cream is an amount of about 1 to about 10 g.

17. The method of claim 11, wherein a unit dose of the vaginal cream is an amount of about 2 to about 6 g.

18. The method of claim 1, wherein the non-infectious inflammatory vulvovaginitis comprises at least one condition selected from the group consisting of erosive vaginitis, mucous membrane pemphigoid, lichen planus, lichen sclerosus, sterile vaginitis, desquamative inflammatory vaginitis, vulvar vestibulitis, autoimmune vaginitis, vaginal blistering disorder and idiopathic inflammatory vaginitis.

19. The method of claim 1, wherein the non-infectious inflammatory vulvovaginitis comprises desquamative inflammatory vaginitis.

20. A method for treating non-infectious inflammatory vulvovaginitis, the method comprising administering to a vulvovaginal surface a pharmaceutical composition comprising clindamycin or a pharmaceutically acceptable salt or ester thereof, wherein the composition has at least one non-lipoidal internal phase and at least one lipoidal external phase that is bioadhesive to the vulvovaginal surface.

21. The method of claim 20, wherein the composition comprises clindamycin or a pharmaceutically acceptable salt or ester thereof in an amount of about 75 to about 400 mg clindamycin equivalent per unit dose.

22. The method of claim 20, wherein the composition comprises clindamycin or a pharmaceutically acceptable salt or ester thereof in an amount of about 125 to about 400 mg clindamycin equivalent per unit dose.

23. The method of claim 20, wherein the composition comprises clindamycin or a pharmaceutically acceptable salt or ester thereof in an amount of about 125 to about 200 mg clindamycin equivalent per unit dose.

24. The method of claim 20, wherein the vulvovaginal surface to which the composition is administered is a vaginal mucosal surface, and the composition is in a form of a vaginal cream having at least one non-lipoidal internal phase and at least one lipoidal external phase that is bioadhesive to the vaginal mucosal surface.

25. The method of claim 24, wherein the vaginal cream comprises clindamycin or a pharmaceutically acceptable salt or ester thereof in a clindamycin equivalent amount of about 1% to about 8% by weight.

26. The method of claim 20, wherein the non-infectious inflammatory vulvovaginitis comprises at least one condition selected from the group consisting of erosive vaginitis, mucous membrane pemphigoid, lichen planus, lichen sclerosus, sterile vaginitis, desquamative inflammatory vaginitis, vulvar vestibulitis, autoimmune vaginitis, vaginal blistering disorder and idiopathic inflammatory vaginitis.

27. The method of claim 20, wherein the non-infectious inflammatory vulvovaginitis comprises desquamative inflammatory vaginitis.

28. A method for treating non-infectious inflammatory vulvovaginitis in a patient, the method comprising:
(a) administering to a vulvovaginal surface of the patient a pharmaceutical composition that comprises clindamycin or a pharmaceutically acceptable salt or ester thereof, wherein the composition is bioadhesive to the vulvovaginal surface; and
(b) administering to the patient a pharmaceutical composition that comprises a steroid compound selected from the group consisting of corticosteroids and hormonal steroids.

29. The method of claim 28, wherein the composition of step (a) comprises at least one non-lipoidal internal phase and at least one lipoidal external phase.

30. The method of claim 29, wherein the composition of step (a) is administered to a vaginal mucosal surface, and is in a form of a vaginal cream having a lipoidal external phase that is bioadhesive to the vaginal mucosal surface.

31. The method of claim 28, wherein the composition of step (b) is administered orally, transdermally, parenterally, subcutaneously, intravenously, intramuscularly, intraperitoneally, buccally, intravaginally, by inhalation, by depot injection, or by hormonal implant.

32. The method of claim 28, wherein the composition of each of steps (a) and (b) comprises at least one non-lipoidal internal phase and at least one lipoidal external phase that is bioadhesive to a vulvovaginal surface.

33. The method of claim 32, wherein steps (a) and (b) occur at the same time and the clindamycin and the steroid compound are administered in a single composition.

34. The method of claim 33, wherein the single composition is in a form of a vaginal cream and is administered to a vaginal mucosal surface.

35. The method of claim 28, wherein the steroid compound comprises a hormonal steroid.

36. The method of claim 28, wherein the non-infectious inflammatory vulvovaginitis comprises at least one condition selected from the group consisting of erosive vaginitis, mucous membrane pemphigoid, lichen planus, lichen sclerosus, sterile vaginitis, desquamative inflammatory vaginitis, vulvar vestibulitis, autoimmune vaginitis, vaginal blistering disorder and idiopathic inflammatory vaginitis.

37. The method of claim 28, wherein the non-infectious inflammatory vulvovaginitis comprises desquamative inflammatory vaginitis.

38. A pharmaceutical composition comprising:
(a) clindamycin or a pharmaceutically acceptable salt or ester thereof in a clindamycin equivalent amount of about 2.5% to about 4% by weight; and
(b) a phospholipid or non-ionic ester surfactant;
wherein the composition is a vaginal cream having at least one non-lipoidal internal phase and at least one lipoidal external phase that is bioadhesive to a vaginal mucosal surface.

39. The composition of claim 38, comprising a non-ionic ester surfactant.

40. The composition of claim 39, that is substantially free of phospholipid.

41. A pharmaceutical composition comprising:
(a) clindamycin or a pharmaceutically acceptable salt or ester thereof in a clindamycin equivalent amount of about 2.5% to about 8% by weight; and
(b) a non-ionic ester surfactant;
wherein the composition is a vaginal cream having at least one non-lipoidal internal phase and at least one lipoidal external phase that is bioadhesive to a vaginal mucosal surface; and wherein the composition is substantially free of phospholipid.

42. A pharmaceutical composition comprising:
(a) clindamycin or a pharmaceutically acceptable salt or ester thereof in a clindamycin equivalent amount of about 2.5% to about 8% by weight; and
(b) a steroid compound selected from the group consisting of corticosteroids and hormonal steroids;
wherein the composition is a vaginal cream having at least one nonlipoidal internal phase and at least one lipoidal external phase that is bioadhesive to a vaginal mucosal surface.

43. A vaginal clindamycin delivery system comprising a unit dosage amount of the vaginal cream composition of claim 38, and a disposable applicator therefor, wherein the applicator is prefilled with a unit dose amount of the composition.

44. A vaginal clindamycin delivery system comprising a unit dosage amount of the vaginal cream composition of claim 41, and a disposable applicator therefor, wherein the applicator is prefilled with a unit dose amount of the composition.
